(19) United States
(12) Patent Application Publication
(10) Pub. No.: US 2021/0104333 A1
(43) Pub. Date: Apr. 8, 2021

(54) TOOL FOR PREDICTING HEALTH AND DRUG ABUSE CRISIS

(71) Applicant: Millennium Health, LLC, San Diego, CA (US)

(72) Inventors: Angela Huskey, San Diego, CA (US); John Penn Whitley, San Diego, CA (US)

(73) Assignee: Millennium Health, LLC, San Diego, CA (US)

(21) Appl. No.: 17/026,042

(22) Filed: Sep. 18, 2020

Related U.S. Application Data
(60) Provisional application No. 62/902,259, filed on Sep. 18, 2019.

(51) Int. Cl. G16H 50/80; G16H 10/40; G06Q 30/00; G16H 10/60; G16H 40/20; G16H 20/10; G16H 50/70; G16H 50/20; G06Q 30/02; G06Q 50/26; G06Q 10/06; G16H 50/30; G06Q 40/08; G06N 20/00; A61B 5/00

(52) U.S. Cl. CPC G16H 50/80; A61B 10/007; G06Q 30/018; G16H 10/60; G16H 40/20; G16H 20/10; G16H 50/70; G16H 50/20; G06Q 30/0205; G06Q 50/26; G06Q 10/06315; G16H 50/30; G06Q 40/08; G06N 20/00; A61B 5/4845; G16H 10/40

(57) ABSTRACT

Systems and methods are provided for understanding, forecasting, managing, and mitigating healthcare crises. A real-time health crisis forecast system and method may include predictor variable data sets such as urine drug testing (UDT) data and demographic data for selected regional populations during selected timeframes and dependent variable data such as mortality rates for selected regional populations during selected timeframes. A health forecast model describing the relationship between the predictor variable and dependent variable data may be generated using selected statistical methods. A model may be used to generate a real-time health crisis forecast for a selected population during a selected timeframe based on inputs of updated predictor variable data. A dashboard presenting graphical representations of a real-time health crisis forecast may provide relevant organizations with a resource allocation and deployment plan, enabling a proactive response.

### CROSS-REFERENCE TO RELATED APPLICATIONS

This application claims the benefit of U.S. Provisional Application No. 62/902,259 filed Sep. 18, 2019 and which is hereby incorporated herein by reference in its entirety.

### TECHNICAL FIELD

The present disclosure relates generally to the use of machine-learning to predictively understand, forecast, manage, and mitigate healthcare crises, including drug abuse and disease spread within localized regions.

### BACKGROUND

Forecasting the localized impact of healthcare crises, such as drug abuse and disease spread, is an important step to pro-actively manage and mitigate such crises. For example, the ability to mitigate the impact of an epidemic, such as the COVID-19 epidemic, may be improved through more accurate prediction of localized disease spread and morbidity. The same is true for mitigating the spread of addiction, for example, to opioids and/or other drugs. In particular, drug overdose death is a growing problem in the United States. 71,000 drug overdose deaths occurred in 2019. More than 36,000 of these deaths were associated with synthetic opioids, such as fentanyl and fentanyl analogs. Studying and understanding drug use trends is critical and essential to saving lives. However, change in trends is currently outpacing the available analytical methods. Prediction of future use trends and identification of areas likely to experience heightened distress is a difficult task due to inconsistent, incorrect, and lagging data.

Existing methods for studying drug crises may assist in understanding past trends but are ill-suited to prediction. Currently, most methods of understanding drug use trends rely on mortality data. Mortality data had limited predictive value, however, because it captures only an endpoint drug use outcome. Therefore, it provides only an indirect measure of changes in drug use because only a small subset of active substance users will overdose and die within a given time period. For instance, in 2018, approximately 32 million Americans had used an illicit drug within the past month and 53 million Americans had used an illicit drug within the past year. These numbers far exceed the number of measured overdose deaths.

Additionally, mortality data introduces significant lag. First, it cannot be measured until the endpoint of the drug use, which may occur following several years or even a lifetime of substance use. Second, mortality data is not collected in a timely fashion or in a consistent fashion across regions of the country. In many cases, by the time sufficient mortality data has been collected to allow for an understanding of changes in drug use trends, months or years have passed, making it difficult or impossible to implement a proactive, life-saving response.

Other types of data, including pre- and post-mortem toxicology data, crime lab data (drug seizure data), household surveys, and emergency room visitation data may have some predictive value but are currently difficult to use for predictive purposes, because the data is not collected consistently across regions, or in a timely fashion.

Many past studies attempting to create a mortality prediction model suffered from similar defects. As discussed above, mortality data has limited predictive value because it only captures an endpoint of drug use and is not always timely updated. Additionally, mortality data, as well as other types of potentially useful data, have limited predictive valuable because they cannot be generalized across populations. In past studies, data sets such as crime lab data or social media scrubbing were used in an attempt to create predictive mortality models, however the data sets were limited to specific geographic areas such as states or counties. Insufficient data in other geographic areas prevented creating a broad model which could be used to provide a comparative risk assessment or resource allocation plan. Additionally, many data sets are only available in urban areas, with dense populations. Since many rural areas have been significantly impacted by drug crises in recent years, data sets with consistency across a broader geographic scope are needed to create valuable predictive models.

Additionally, as is the case for mortality data in many counties, other types of data, including demographic data, may only be updated on an annual basis. Data that is not updated in a timely fashion is difficult to rely on to formulate valuable predictive models. Data that is collected and updated on a monthly, weekly, or even daily basis would enable modeling with far more predictive value.

Therefore, use of a data source or sources that are collected in a timely fashion, updated frequently, collected consistently across regions, and collected in both urban and rural areas is essential for real-time drug crisis prediction. A valuable real-time prediction framework needs two key elements. First, it needs an input of up to date data, without the lag problems discussed above, so that it can enable users to take action before a crisis occurs. Second, it needs sufficient data, collected consistently across geographic regions, such that comparative predictions can be made for subregions within a region. For instance, a framework with high predictive value could enable a state government to predict and compare the risk among all counties within the state. Current methods largely fail to meet both of these key criteria.

### BRIEF SUMMARY

The disclosure relates to use of statistical modeling techniques to correlate and predict health crises, using carefully selected data to resolve the timeliness and consistency problems discussed above. Examples of health crises may include drug overdose crises as well as the spread of disease in a localized region. In one embodiment, clinical urine drug testing (“UDT”) positivity rates may be used to develop a model for drug overdose mortality rates. A positive rate refers to the rate of number of UDT tests indicating the presence of drugs over the total number of UDT tests conducted. In another embodiment, demographic covariates, such as employment rates, education rates, poverty rates, and insurance rates, may be used to create a model. In another embodiment, both UDT data and selected demographic covariates may be used to create a model.

Temporal and spatial parameters may be selected for the drug crisis prediction model. For example, a mortality rate may be estimated at the national, state, county, or city level. A mortality rate may also be estimated at the individual zip code level. The time scale for the mortality estimate may be updated on a yearly, quarterly, or even monthly schedule. The mortality data may also vary by specific cause of death. For instance a model may include mortality data for cases of suicide or accident. The model may also be limited to mortality data associated with a specific class or classes of drugs, such as fentanyl. UDT data used to create a model may also have temporal and spatial parameters. For instance, UDT data relied upon may be collected at the yearly, monthly, weekly, or even daily level. UDT data can also be collected at the state, county, city, or zip code level.

The embodiments discussed below relate to modeling of yearly overdose deaths collected for U.S. counties as a function of UDT positivity rates and other selected demographic characteristics. In some embodiments, counties with fewer than 10 deaths in any given year may be eliminated from the model, in accordance with the Center for Disease Control and Prevention (CDC)'s guidance. Counties with fewer than 10 UDT tests in a given year may also be eliminated.

UDT data may be useful in monitoring drug use trends because it includes desirable characteristics, namely, it is collected and updated in a timely fashion, and it is collected as a part of routine medical care and thus consistently available across geographic regions. In some embodiments, a drug crisis prediction model may be generated by empirically describing the relationship of UDT data to overdose mortality estimates. Such a model may have high predictive value because it relies on timely and generalizable data. The model may then be useful in not only understanding drug use trends but predicting trends at a monthly, weekly, or even daily level. A timely, predictive model may provide a forecasting capability that could provide advance warnings for regions likely to experience drug use stress. The predictions could be generated at a state, county, city, or even zip code level. Advanced forecasting capabilities may allow relevant organizations to plan for and even avert a potential drug crisis through efficient resource allocation and deployment strategies. Relevant organizations may include harm reduction services, first responders, law enforcement, scientific research organizations, the medical community, and policy makers. Communicating a drug crisis prediction to these organizations may allow for a more proactive and efficient response which may save lives and allow for better and more accurate understanding of current drug use trends.

In an embodiment of the present disclosure, a health forecasting system may include a health forecasting logical circuit and a graphical user interface. The health forecasting logical circuit may comprise a processor. The health forecasting system may also include a non-transient memory having embedded computer executable instructions. The instructions may cause the processor to obtain a first data set form a first data source. The first data set may include positive drug test rates for one or more controlled substances, crime lab seizure data, emergency room visitation data, prescription rates, and demographic data for a regional population. The processor may then obtain a second data set from a second data source. The second data set may include mortality data for a regional population.

The process may train a health forecasting modell. The health forecasting model may describe a relationship between the second data set and the first data set. The model may be trained using a dual validation approach including validation of two temporally offset data sets.

In an embodiment, the health forecasting model may include a logistic regression model, a gradient-boosted decision tree, or a cognitive neural network.

In another embodiment, the processor in a health forecasting system may perform further steps. The processor may update a first data set from a first data source on a selected time interval. The processor may then apply a health forecasting model to the updated first data set. The processor may then generate a real-time health crisis forecast based on the application of the health forecasting model to the updated first data set for a selected time interval. The processor may further generate one or more graphical data representations based on the generated health crisis forecast. The graphical data representations may be based on user-selected model data structures.

In another embodiment, the processor in a health forecasting system may perform further steps. The processor may obtain a third data set from a third data source. The third data set may include available drug crisis response resources for a regional population. The processor may further generate a resource deployment plan based on the health crisis forecast and the available drug crisis response resources in a selected geographical region.

In another embodiment, the drug crisis system may further generate comparative drug crisis forecasts for multiple selected geographic regions. These comparative forecasts may form the basis for a comparative risk assessment. The system may further generate a resource allocation plan for the selected geographic regions based on the comparative risk assessment.

In an embodiment, the first data set may include urine drug testing (UDT) data. In another embodiment, the first data set may include demographic data, which may include unemployment rates, education rates, poverty rates, and insurance rates. In an embodiment, UDT data may be collected at the county level for a regional population and may be updated on a monthly timeframe. In an embodiment, may be collected at the county level and may be updated on a monthly timeframe.

In an embodiment, the processor may use one or more regression methods to train the health forecasting model, including Poisson regression, negative binomial regression, logistic regression, regression trees, random forest, regularized regression, and non-linear prediction.

In an embodiment, the user-selected model data structures may provide a comparative risk assessment and may include a heat map or a table ranking counties by determined risk level.

In an embodiment of the present disclosure, a health forecasting method may include obtaining a first data set from a first data source using a graphical user interface. The first data set may include positive drug test rates for one or more controlled substances, crime lab seizure data, emergency room visitation data, prescription rates, and demographic data for a regional population. The method may further include obtaining a second data set from a second data source with a graphical user interface. The second data set may include mortality data for a regional population.

The method may further include training a health forecasting model using a dual validation approach including temporally offset data sets. The health forecasting model may be trained to describe a relationship between the first and second data sets.

In an embodiment, the health forecasting model may include a logistic regression model, a gradient-boosted decision tree, or a cognitive neural network.

The method may further include updating the first data set form the first data source on a selected time interval. The method may further include generating a real-time health crisis forecast based on the application of the health forecasting model to the updated first data set for a selected time interval. The method may further include generating one or more graphical data representations based on the generated health crisis forecast. The graphical data representations may be based on user-selected model data structures.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1A is a flowchart of a method for training model parameters to describe a relationship between a predictor variable data set and a dependent variable data set.

FIG. 1B is a flowchart of a method for obtaining user input in selecting a first predictor variable data set.

FIG. 1C is a flowchart of a method for obtaining user input in selecting a dependent variable data set.

FIG. 1D is a flowchart of a method for obtaining user input in training model parameters to describe a relationship between a first and second data set.

FIG. 1E is a flowchart of a method for generating a real-time drug crisis forecast.

FIG. 1F is a flowchart of a method for a subprocess for generating graphical representations of a real-time drug crisis.

FIG. 1G is a flowchart of a method for generating a resource deployment plan based on a generated real-time drug crisis forecast.

FIG. 1H is a flowchart of a method for obtaining user input in selecting a third data set comprising response resource data.

FIG. 1I is a flowchart of a method for obtaining user input in generating a resource deployment plan.

FIG. 2 illustrates an example drug crisis prediction system.

FIG. 3 illustrates an example resource deployment communication system.

FIG. 4 illustrates an example of a drug crisis predictor and dependent variable database for use in drug crisis prediction systems and methods.

FIG. 5 illustrates an example of a graphical representation of a real-time drug crisis forecast comprising a table.

FIG. 6 illustrates an example of a graphical representation of a real-time drug crisis forecast comprising a choropleth map.

FIG. 7 illustrates an example computing component that may be used to implement features of various embodiments of the disclosure.

The figures are not exhaustive and do not limit the present disclosure to the precise form disclosed.

### DETAILED DESCRIPTION

Some embodiments of the disclosure provide a method for generating a real-time health crisis forecast based on careful selection of predictive data and modeling to empirically develop a predictive model describing the relationship between predictive data and mortality data. For example, Urine Drug Testing (UDT) data may be selected for a specific time frame to create a predictive model. For example, UDT data may be collected for about a six year period, from the years 2013 to 2018. UDT results of patient specimens submitted for testing by health care professionals as part of routine care may be used. Specimens may be collected for the entire country. Specimens may also be collected for a geographic subregion, such as a specific state or county. A single specimen for each patient may be selected based on the earliest specimen collection date and may be used for downstream analysis. This selection may be performed to remove repeated measurements for the same patient from the analysis.

While the prediction model is described herein with reference, for example, to forecasting and understanding drug use, it should be understood that the same model may also be applied to forecasting disease spread across local regions to manage a disease and/or infection crisis, and/or mitigate the localized impact of an epidemic, such as the COVID-19 epidemic.

Because UDT data may be collected during routine medical care, a large sample size may be used to create an accurate predictive model. For instance, in one example embodiment, a sample size exceeding 1 million randomly sampled patient specimens may be used. Samples may be collected for adult patients. The UDT tests selected for inclusion may test for several classes of drugs including methamphetamine, heroin, fentanyl and prescription opioids. The UDT tests may employ a liquid chromatography-tandem mass spectrometry method to detect the presence of drugs in selected drug classes. The liquid chromatography-tandem mass spectrometry testing method is a laboratory-developed test with performance characteristics determined by Millennium Health, San Diego, Calif., which is certified by the Clinical Laboratory Improvement Amendments and accredited by the College of American Pathologists for high-complexity testing.

UDT tests may be used to identify at least the following classes of drugs: methamphetamine, cocaine (benzoylecgonine), fentanyl (fentanyl and norfentanyl), heroin (6-MAM) and prescription opioids (codeine, hydrocodone, norhydrocodone, hydromorphone, morphine, oxycodone, noroxycodone and oxymorphone). A UDT test result may be considered positive if any parent analyte or metabolite within a drug class is detected. In some embodiments, in the prescription opioid class, all analytes and/or metabolites may need to be ordered and a valid testing result of all analytes and/or metabolites may be necessary for each specimen in order to accurately confirm an affirmative finding that drugs in the opioid class were detected. Health care professionals may report a patient's prescribed medications and disclose that information along with a UDT test. In some embodiments, UDT results analyzed may only include non-prescription drugs.

An embodiment including the study of UDT data may follow a study protocol approved by an appropriate body, such as the Aspire Independent Review Board. For example, consistent with best practices, the study of UDT data may include a waiver of consent for the use of deidentified patient data and may conform to study guidelines set forth in the Strengthening the Reporting of Observational Studies in Epidemiology (STROBE) reporting guideline.

In some embodiments, mortality data indicating drug overdose deaths may be identified in the National Vital Statistics System multiple cause-of-death mortality files using International Classification of Diseased, Tenth Revision (ICD-10) or another appropriate data source. In some embodiments, underlying cause-of-death codes (UCD) including X40-X44 (unintentional), X60-X64 (suicide), X85 (homicide), and Y10-Y14 (undetermined intent) may be considered and collected to estimate drug overdose mortalities. In some embodiments, deaths having drug overdose identified as the underlying cause of death may be included in the mortality data set used to create a model if the following ICD-10 multiple cause-of-death (MCD) codes were indicated: T40.1 (heroin), T40.2 (natural/semisynthetic opioids), T40.4 (other synthetic narcotics), T40.5 (cocaine), and T43.6 (psychostimulants with abuse potential). Mortality rates may be collected at a selected regional level. For instance, In some embodiments, mortality rates may be collected at the state level. In another embodiment, mortality rates may be collected at the county level. In another embodiment, mortality rates may be collected at both the state and county level and/or for some other regional population. Mortality rates may be collected for a selected time interval. In some embodiments, mortality rates may be collected over a recent five-year interval.

In some embodiments, demographic data for regional populations may be collected form the American Community Survey (ACS 2018 Data Release) and/or from another appropriate data source. Regional population features having predictive value may be selected from the demographic data. Demographic data may be collected for a selected time interval. In some embodiments, demographic data may be collected over a recent five-year interval. Demographic data may be collected over a time interval of about 5 years. In some embodiments, demographic data may be collected at the state, county, or city level, or for any other selected geographic region or regions of interest. In some embodiments, selected data may include social, economic, housing, and/or demographic data obtained from the ACS data profiles.

In some embodiments, other demographic data, such as annual U.S. opioid prescription rates may be collected for inclusion in the model. This data may be collected from the Center for Disease Control and Prevention (CDC) or from another appropriate data source. This data may be collected on a selected time interval. For example the data may be collected for a specific year, such as the year 2018. The data may be collected for selection regional populations, such as at the state and county level. In some instances, data may be missing or insufficient for a selected regional population, such as a particular county, for a selected time interval, such as during a given year. In these instances, the missing or insufficient data rates may be imputed using a mean level for an encompassing geographic region, for instance at the state level, for the selected year. For example, Table 1, below, shows demographic features which may be selected for inclusion in a model and shows an imputed prescription rate feature for a selected year:

TABLE 1
Embodiment Showing Selected Demographic Features
for Inclusion in a Predictive Model

| ACS variable | ID | Model ID |
|---|---|---|
| DP05_0001E | Estimate!!SEX | total_population. (used as |
| | AND AGE!!Total | poisson offset in model) |
| | population | |
| DP05_0002PE | Percent!!SEX AND | pct_male |
| | AGE!!Male | |
| DP05_0017E | Estimate!!SEX | median_age |
| | AND AGE!!Median | |
| | age (years) | |
| DP05_0059PE | Percent!!RACE!!White | pct_white |
| DP03_0009PE | Percent!!EMPLOYMENT | pct_unemployed |
| | STATUS!!Percent | |
| | Unemployed | |
| DP03_0062E | Estimate!!INCOME | median_householdIncome |
| | AND BENEFITS | |
| | (IN 2012 INFLATION- | |
| | ADJUSTED | |
| | DOLLARS)!!Median | |
| | household income | |
| | (dollars) | |
| DP03_0099PE | Percent!!HEALTH | pct_noHealthInsurance |
| | INSURANCE | |
| | COVERAGE!!No | |
| | health insurance | |
| | coverage | |
| DP03_0119PE | Percent!!PERCENTAGE | pct_familyBelowPoverty |
| | OF FAMILIES AND | |
| | PEOPLE WHOSE | |
| | INCOME IN THE | |
| | PAST 12 MONTHS | |
| | IS BELOW THE | |
| | POVERTY LEVEL!!All | |
| | families | |
| DP02_0067PE | Percent!!EDUCATIONAL | pct_higherEdGrad |
| | ATTAINMENT!!Percent | |
| | bachelor's degree or | |
| | higher | |
| DP02_0069PE | Percent!!VETERAN | pct_veterans |
| | STATUS!!Civilian | |
| | veterans | |
| DP02_0071PE | Percent!!DISABILITY | pct_disability |
| | STATUS OF THE | |
| | CIVILIAN | |
| | NONINSTITUTIONALIZED | |
| | POPULATION!!With | |
| | a disability | |
| DP02_0009PE + | Percent single male + | pct_singleHousehold |
| DP02_0007PE | single female head of | |
| | household | |
| NA | County leevl opiod | mean_imputed_Prescribing_Rate |
| | prescription rate | |
| | (missing county | |
| | data imputed with | |
| | the state average | |
| | for a given year) | |
| NA | UDT positivity rate | Methamphetamine Positivity (%) |
| | in percentage | |
| NA | UDT positivity rate | Cocaine Positivity (%) |
| | in percentage | |
| NA | UDT positivity rate | Heroin Positivity (%) |
| | in percentage | |
| NA | UDT positivity rate | Fentanyl Positivity (%) |
| | in percentage | |

A statistical method or methods may be selected to create a predictive model. In some embodiments, a regression model, such as a Poisson regression, may be used to generate a predictive model. The dependent variable in an example embodiment using a Poisson regression may be the number of drug overdose deaths occurring in a selected geographic region, such as a county, during a selected time interval, such as a given year. Predictor variables in an example embodiment using a Poisson regression may be UDT positive rates and/or data comprising selected demographic features for a selected geographic region, such as the county or state level, during a selected time interval. Table 1, above, shows selected predictor variables for inclusion in an model in an example embodiment.

In some embodiments, an initial regression model may be used in which the regression model treat the county and state as random variables in a random intercept model. County may be nested within state. Fixed factor regression coefficients may be converted to incidence rate ratios (IRR) to allow for easier interpretation of the modeled relationship. Mortality and incidence rates may be determined as deaths per 100,000 members of a population. Statistical models may be determined on estimates for a selected geographic region, such as the county level, and for a selected time interval, such as over a recent five-year period.

In some embodiments, validations may be performed to evaluate the predictive value of the generated model. In some embodiments, a validation may train Poisson model parameters with county level data over a two year period and a test dataset from a year preceding the two year training data may be used. A second validation using data shifted forward an additional year may be used. Performance metrics used to assess the accuracy and predictive value of the model may be used. These may include a Pearson correlation of observed and predicted mortality rates, a Mean Square Error (MSE), a Mean Absolute Deviation (MAD), and/or a Mean Absolute Percent Error (MAPE). Statistical software, such as R statistical software version 4.0.2 (R Project for Statistical Computing) or another appropriate program or method may be used for data analysis. In some embodiments using R for data analysis, the glmr( ) function from the Ime4 v1.1-23 package may be used for estimating Poisson mixed models. In other embodiments, other appropriate estimate methods may be used. Table 2, below, shows an example embodiment of pairwise univariate Pearson correlations for a morality rate and other predictor variables determined for a regression model for a selected year:

TABLE 2
Embodiment Showing Univariate Correlation of Mortality and Predictor Variables Determined for a Given Year at the County Level

| Pearson Correlation | pct_singleHousehold | pct_higherEdgrad | pct_veterans | pct_disability | pct_unemployed |
|---|---|---|---|---|---|
| pct_singleHousehold | 1.0000 | −0.4748 | 0.0084 | 0.2047 | 0.5901 |
| pct_higherEdgrad | −0.4748 | 1.0000 | −0.3608 | −0.7316 | −0.5256 |
| pct_veterans | 0.0084 | −0.3608 | 1.0000 | 0.4189 | 0.0699· |
| pct_disability | 0.2047 | −0.7316 | 0.4189 | 1.0000 | 0.4875 |
| pct_unemployed | 0.5901 | −0.5256 | 0.0699 | 0.4875 | 1.0000 |
| pct_householdincome | −0.5172 | 0.7299 | −0.2062 | −0.7475 | −0.5630 |
| pct_noHealthinsurance | 0.4488 | −0.3022 | 0.0184 | 0.1257 | 0.3769 |
| pct_familyBelowPoverty | 0.7466 | −0.5422 | −0.0632 | 0.5024 | 0.7273 |
| pct_male | −0.1109 | −0.1869 | 0.2428 | 0.0270 | −0.0318 |
| median_age | −0.4989 | −0.1320 | 0.1955 | 0.3738 | −0.0931 |
| pct_white | −0.4971 | −0.1998 | 0.2166 | 0.2775 | −0.3691 |
| mean_imputed_Prescribing_Rate | 0.1005 | −0.5638 | 0.4276 | 0.6984 | 0.2042 |
| methamphetamine_positivity | 0.0349 | −0.1096 | 0.0673 | 0.0839 | −0.0624 |
| heroin_positivity | −0.0303 | 0.0530 | −0.0636 | −0.0199 | −0.0594 |
| cocaine_positivity | 0.0407 | 0.0665 | −0.0703 | 0.0187 | 0.0230 |
| fentanyl_positivity | −0.0512 | −0.0058 | −0.0205 | 0.0280 | −0.0807 |
| opioids_positivity | −0.0450 | −0.1799 | 0.0325 | 0.1861 | 0.0604 |
| Crude.Rate | 0.0159 | −0.3266 | 0.1538 | 0.4644 | 0.1188 |

| Pearson Correlation | pct_householdincome | pct_noHealthinsurance | pct_familyBelowPoverty | pct_male |
|---|---|---|---|---|
| pct_singleHousehold | −0.5172 | 0.4488 | 0.7446 | −0.1109 |
| pct_higherEdgrad | 0.7299 | −0.3022 | −0.5422 | −0.1869 |
| pct_veterans | −0.2062 | 0.0184 | −0.0632 | 0.2428 |
| pct_disability | −0.7475 | 0.1257 | 0.5024 | 0.0270 |
| pct_unemployed | −0.5630 | 0.3769 | 0.7273 | −0.0318 |
| pct_householdincome | 1.0000 | −0.4068 | −0.7702 | 0.0992 |
| pct_noHealthinsurance | −0.4068 | 1.0000 | 0.5700 | −0.0075 |
| pct_familyBelowPoverty | −0.7702 | 0.5700 | 1.0000 | −0.1305 |
| pct_male | 0.0992 | −0.0075 | −0.1305 | 1.0000 |
| median_age | 0.0054 | −0.2887 | −0.3020 | −0.1708 |
| pct_white | −0.0461 | −0.3250 | −0.3502 | 0.3077 |
| mean_imputed_Prescribing_Rate | −0.5496 | 0.0957 | 0.2407 | −0.0510 |
| methamphetamine_positivity | −0.0850 | 0.0943 | 0.0535 | 0.2288 |
| heroin_positivity | 0.0910 | −0.2132 | −0.0588 | −0.0134 |
| cocaine_positivity | −0.0187 | −0.1774 | 0.0229 | −0.2536 |
| fentanyl_positivity | 0.0435 | −0.3256 | −0.0932 | −0.1401 |
| opioids_positivity | −0.0384 | −0.0662 | 0.0223 | −0.0025 |
| Crude.Rate | −0.2663 | −0.1936 | 0.1361 | −0.0412 |

| Pearson Correlation | median_age | pct_white | mean_imputed_Prescribing_Rate | methamphetamine_positivity | heroin_positivity |
|---|---|---|---|---|---|
| pct_singleHousehold | −0.4989 | −0.4971 | 0.1005 | 0.0349 | −0.0303 |
| pct_higherEdgrad | −0.1320 | −0.1998 | −0.5638 | −0.1096 | 0.0530 |
| pct_veterans | 0.1955 | 0.2166 | 0.4276 | 0.0673 | −0.0636 |
| pct_disability | 0.3738 | 0.2775 | 0.6984 | 0.0839 | −0.0199 |
| pct_unemployed | −0.0931 | −0.3691 | 0.2042 | −0.0624 | −0.0594 |
| pct_householdincome | 0.0054 | −0.0461 | −0.5496 | −0.0850 | 0.0910 |
| pct_noHealthinsurance | −0.2887 | −0.3250 | 0.0957 | 0.0943 | −0.2132 |
| pct_familyBelowPoverty | −0.3020 | −0.3502 | 0.2407 | 0.0535 | −0.0588 |
| pct_male | −0.1708 | 0.3077 | −0.0510 | 0.2288 | −0.0134 |
| median_age | 1.0000 | 0.4671 | 0.2727 | −0.1885 | 0.0579 |
| pct_white | 0.4671 | 1.0000 | 0.3065 | 0.1200 | 0.0649 |
| mean_imputed_Prescribing_Rate | 0.2727 | 0.3065 | 1.0000 | 0.1395 | −0.0345 |
| methamphetamine_positivity | −0.1885 | 0.1200 | 0.1395 | 1.0000 | 0.1349 |
| heroin_positivity | 0.0579 | 0.0649 | −0.0345 | 0.1349 | 1.0000 |
| cocaine_positivity | 0.1546 | −0.0742 | −0.0445 | −0.1620 | 0.5483 |
| fentanyl_positivity | 0.1722 | 0.1418 | −0.0201 | −0.0628 | 0.6295 |
| opioids_positivity | 0.1922 | 0.1641 | 0.2179 | 0.0330 | 0.4482 |
| Crude.Rate | 0.3148 | 0.2430 | 0.3088 | −0.0907 | 0.3814 |

| Pearson Correlation | cocaine_positivity | fentanyl_positivity | opioids_positivity | Crude.Rate |
|---|---|---|---|---|
| pct_singleHousehold | 0.0407 | −0.0512 | −0.0450 | 0.0159 |
| pct_higherEdgrad | 0.665 | −0.0058 | −0.1799 | −0.3266 |
| pct_veterans | −0.0703 | −0.0205 | 0.0325 | 0.1538 |
| pct_disability | 0.0187 | 0.0280 | 0.1961 | 0.4644 |
| pct_unemployed | 0.0230 | −0.0807 | 0.0604 | 0.1188 |
| pct_householdincome | −0.0187 | 0.0435 | −0.0384 | −0.2663 |
| pct_noHealthinsurance | −0.1774 | −0.3256 | −0.0662 | −0.1936 |
| pct_familyBelowPoverty | 0.0229 | −0.0932 | 0.0223 | 0.1361 |
| pct_male | −0.2536 | −0.1401 | −0.0025 | −0.0412 |
| median_age | 0.1546 | 0.1722 | 0.1922 | 0.3148 |
| pct_white | −0.0742 | 0.1418 | 0.1641 | 0.2430 |
| mean_imputed_Prescribing_Rate | −0.0445 | −0.0201 | 0.2179 | 0.3088 |
| methamphetamine_positivity | −0.1620 | −0.0628 | 0.0330 | −0.0907 |
| heroin_positivity | 0.5483 | 0.6295 | 0.4482 | 0.3814 |
| cocaine_positivity | 1.0000 | 0.5471 | 0.3066 | 0.3987 |
| fentanyl_positivity | 0.5471 | 1.0000 | 0.3954 | 0.4463 |
| opioids_positivity | 0.3066 | 0.3954 | 1.0000 | 0.2748 |
| Crude.Rate | 0.3987 | 0.4463 | 0.2748 | 1.0000 |

In an embodiment of the present disclosure, a processor may empirically determine a health forecasting model by obtaining training data sets and performing regression modeling. The health forecasting model may be trained by empirically determining the relationship between two training data sets. For example, the training data sets may include a predictor variable data set, including, for example, UDT data and demographic data, and a dependent variable data set, including, for example, mortality data. A regression model may be used to express the relationship describing the dependent variable data, such as mortality data, as a function of the predictor variable data, such as the UDT and demographic data. Coefficients for the regression model may be empirically determined based on the training data sets. Then, a health forecasting model including the empirically determined coefficients can be generated to describe the relationship between predictor and dependent variable data.

In an embodiment, regression models may be used. For example a linear regression model, given by the function:

F(x)=(B₀+B₁x₁+B₂x₂+ . . . +Bₖxₖ)

may be applied to express the dependent variable data as a function of the predictor variable data set. The coefficients B₀, B₁, B₂, . . . , Bₖ may be empirically determined and used to generate a health forecasting model.

In another embodiment, a Poisson regression model, given by the expression:

ln(F(x))=(B₀+B₁x₁+B₂x₂+ . . . +Bₖxₖ)

may be applied, for example, to express a morality rate as a function of a UDT positivity rate and other demographic rates, such as education rates, insurance rates, unemployment rates, and poverty rates. As described above with respect to the linear regression model, coefficients may be empirically determined to by applying the Poisson regression model to training data sets to generate a health forecasting model including the determined coefficients.

Those having skill in the art will appreciate that these functions are merely example functions and other statistical methods may be available. Additionally, correction coefficients and other known modeling concepts may be applied in conjunction with the above regression models, or other models, to accurately derive a health forecasting model.

As shown below in Table 3, Poisson regression coefficients (Incident Rate Ratios), may be determined for a selected test data time interval. For example, the test data time interval may be from about the years 2013 to 2018. For example, a Poisson coefficient may represent the contribution of a specific class of drug detected by UDT tests. For example, as shown below, positive detection of fentanyl has a Poisson coefficient of 1.091 which indicates the relationship this factor has on predicted mortality. A Poisson coefficient of 1.091, in this example, may indicated that for every increase of fentanyl by 1 unit, deaths in the county which the health forecasting model was generated to describe may increase 9.1% in a given year. Poisson coefficients represent the effect of fentanyl positive rates, unemployment rates, disability rates, methamphetamine rates, education rates, opioid rates, poverty rates, heroin rates, prescription rates, and insurance rates, as well as the effect of other predictive factors.

TABLE 3
Embodiment Showing Example Poisson Regression Coefficients
Poisson Regression Coefficients (Incidence Rate Ratios)

| Variable | Est | LL | UL | pval |
|---|---|---|---|---|
| (Intercept) | 0.000155 | 0.000138 | 0.000173 | 0.00E+00 |
| pct_unemployed | 0.694148 | 0.676398 | 0.712364 | 5.91E−168 |
| fentanyl_positivity | 1.09099 | 1.082016 | 1.100038 | 7.14E−95 |
| pct_familyBelowPoverty | 1.302187 | 1.237623 | 1.370119 | 2.51E−24 |
| pct_disability | 1.242781 | 1.190094 | 1.297801 | 8.02E−23 |
| pct_male | 1.2057 | 1.157682 | 1.25571 | 1.86E−19 |
| methamphetamine_positivity | 0.960348 | 0.950655 | 0.970141 | 5.43E−15 |
| median_age | 1.19612 | 1.135697 | 1.259758 | 1.28E−11 |
| pct_higherEdGrad | 1.170343 | 1.103433 | 1.24131 | 1.63E−07 |
| pct_veterans | 0.889224 | 0.850691 | 0.929501 | 2.05E−07 |
| opioids_positivity | 0.97725 | 0.968712 | 0.985862 | 2.74E−07 |
| median_householdIncome | 0.911468 | 0.867975 | 0.95714 | 2.02E−04 |
| heroin_positivity | 0.989904 | 0.981699 | 0.998177 | 1.69E−02 |
| cocaine_positivity | 1.010967 | 1.00157 | 1.020453 | 2.21E−02 |
| pct_singleHousehold | 1.033515 | 0.993647 | 1.074982 | 1.00E−01 |
| mean_imputed_Prescribing_Rate | 1.014594 | 0.989921 | 1.039883 | 2.49E−01 |
| pct_white | 1.011499 | 0.961653 | 1.063928 | 6.57E−01 |
| pct_noHealthInsurance | 0.998021 | 0.969163 | 1.027738 | 8.95E−01 |

In alternative embodiments, other modeling methods may be used to generate a health forecasting model by empirically determining the relationship between training data sets. For example, machine learning techniques, such as a gradient-boosted decision tree, or a cognitive neural network, may be applied to training data sets.

### Example Embodiment for Training Health Forecasting Model

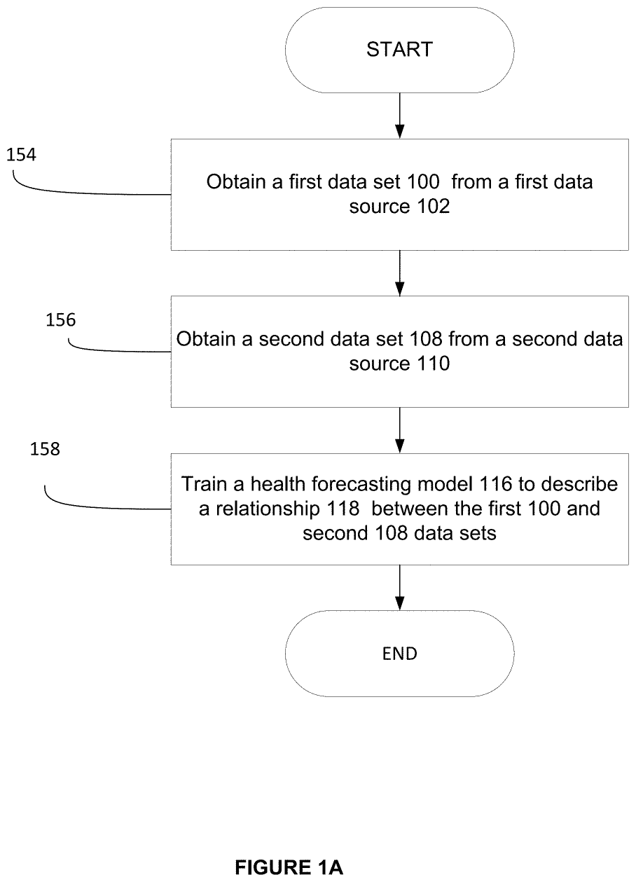

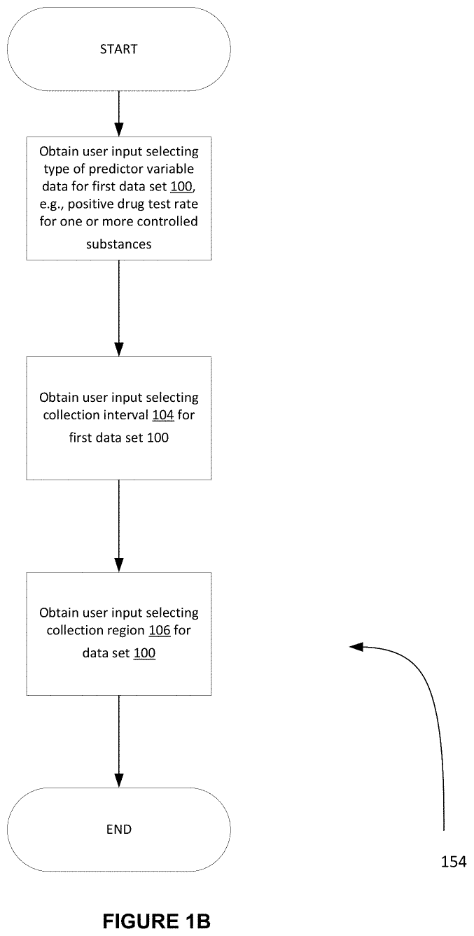

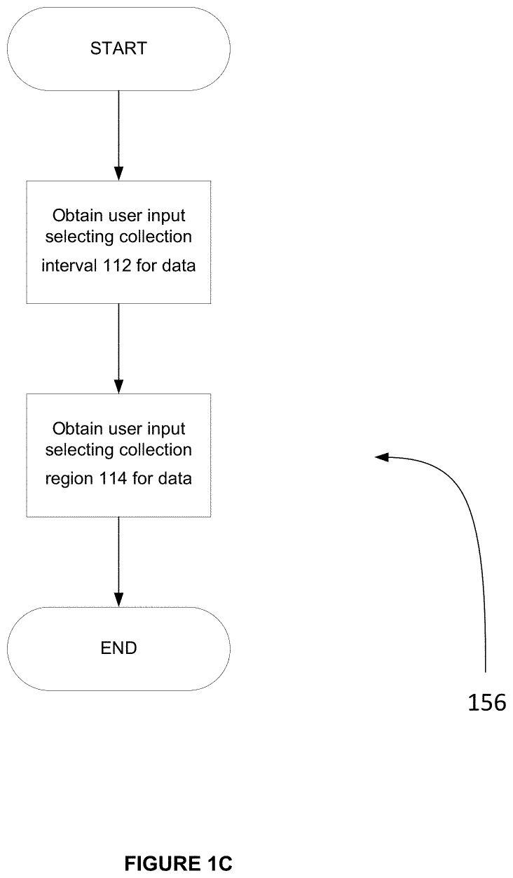

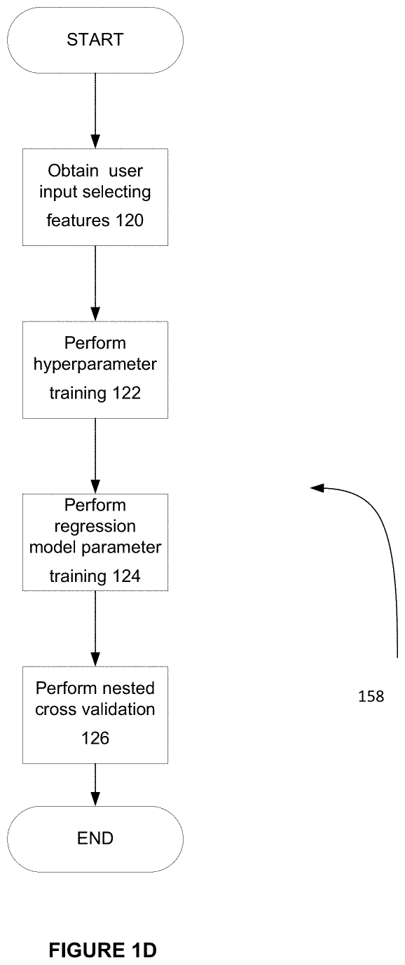

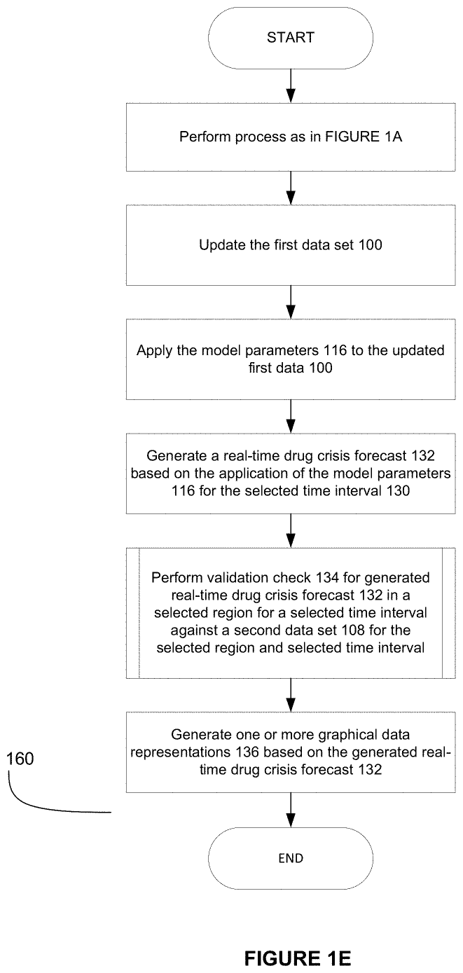

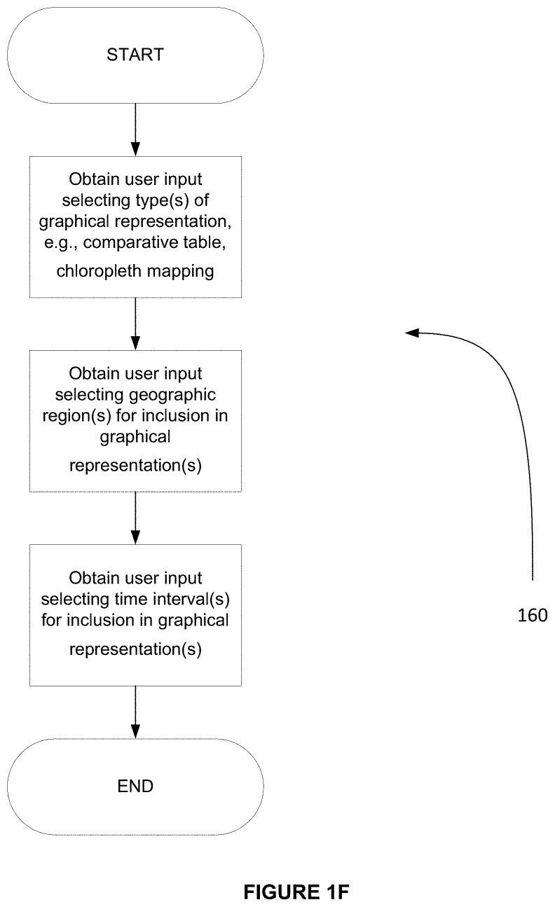

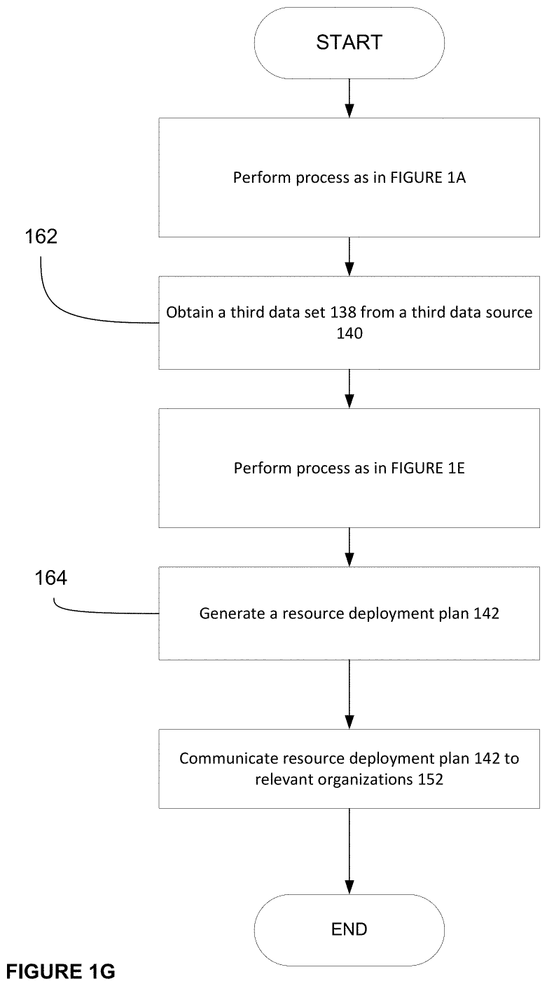

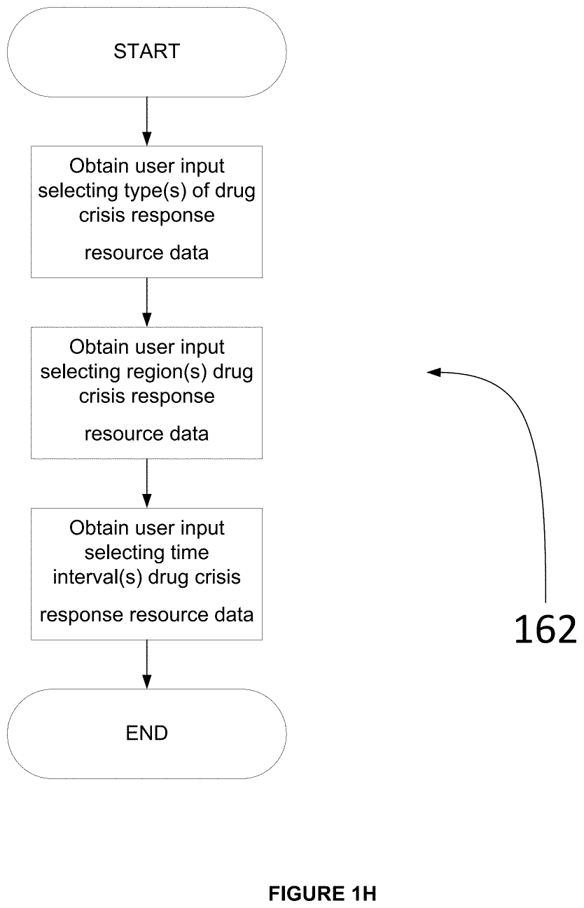

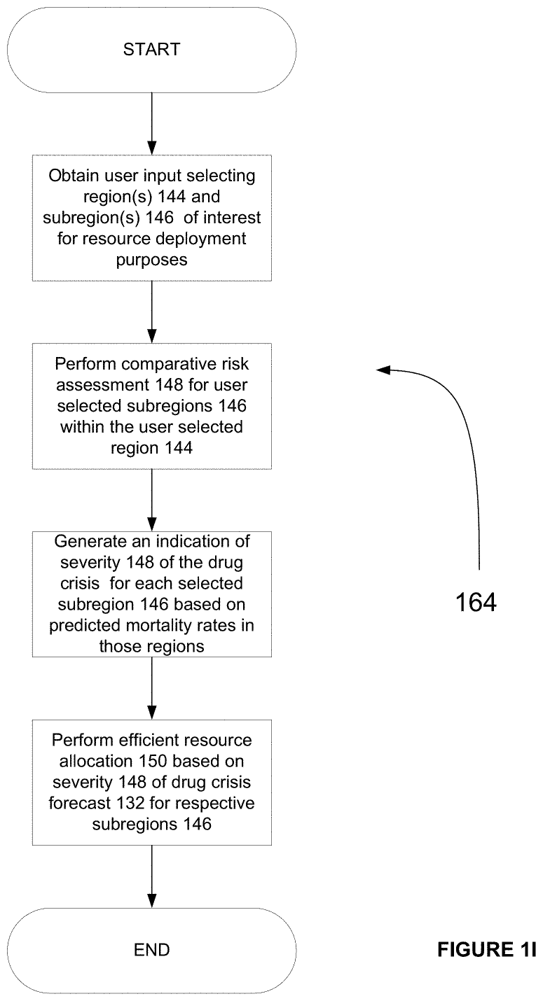

With reference now to FIG. 1A of the illustrative drawings, there is shown a flowchart explaining an example of a method for training a health forecasting model 116 to describe a relationship 118 between selected predictor and independent variables. In the example method of FIG. 1A the processor performs a first step 154 where it obtains a first data set 100 from a first data source 102. The first data set may comprise predictor variables. The first data set 100 may comprise urine drug testing (UDT) positivity rates. The first data set 100 may further comprise selected demographic data. The UDT rates and demographic data may be collected over a selected time interval. The UDT and demographic data may be collected for a selected geographic region or regions. Other predictor variable data may also be obtained and may include crime lab seizure data, wastewater metabolite level data, and social media data. The first data source 102 may comprise census data, household surveys, lab collected data, and other appropriate data sources.

With reference now to FIG. 1B of the illustrative drawings, there is shown a flowchart explaining an example method for performing the first step 154 of the process shown in FIG. 1A. The first step 154 involves obtaining a first data set 100 from a first data source 110. To perform the first step 154, a processor may obtain input from a user which indicates the type(s) of predictor variable data for the first data set 100. The predictor variable data may include a positive drug test rate for one or more controlled substances. In some embodiments, the positive drug test may be a UDT test which detects the presence of several classes of drugs including methamphetamine, heroin, fentanyl and prescription opioids. In another embodiment, the processor may obtain a user selection of predictor variable data comprising UDT positivity rates for only one type of drug, for instance fentanyl. Selecting only one drug type may increase the accuracy of the later generated predictive model. The first data set 100 may also contain user selected demographic features for a regional population. The selected demographic data may include education rates, unemployment rates, poverty rates, prescription rates, insurance rates, and other selected demographic features having predictive value.

A processor may then obtain user input selecting the collection interval 104 for the first data set 100. The collection interval 104 may be at an annual, monthly, weekly, or daily level. For example, a user may selected data for about a two year period. In an example embodiment, a user may selected data for the years 2016-2018. A processor may further obtain user input selecting the collection region 106 for the first data set 100. The selected region(s) 106 may be at the country, state, county, city, or individual zip code level. For example, a user may select UDT data for a specific county. In an example embodiment, the UDT data may be selected for Los Angeles County. In an example embodiment, a user may select a first data set 100 comprising both UDT and demographic data. The UDT data may be collected on monthly time interval 104. The UDT data may be collected for all counties 106 within a state. Other embodiments exist. Some data types may only be available on an annual time interval 104. Some data types may also not be available for every selected county 106 within the selected collection interval 104. In these situations, an embodiment may include imputation of data based on mean values measured for an encompassing collection region. For instance, in one embodiment, where data is not available for a selected county in a given year, data may be imputed based on a state level mean.

In some embodiments, imputation methods may improve performance and generalizability. Imputation methods may include UDT imputation (spatio-temporal smoothing and prediction) to improve coverage and mortality (death) imputation via multiple imputation to impute values onto counties or regions without data.

Referring back to FIG. 1A, in the example method shown, the processor then performs a second step 156 where it obtains a second data set 108 from a second data source 110. The second data set may comprise mortality data related to drug overdose. The mortality data may be selected for a specific cause of death, including unintentional, suicide, homicide, and undetermined intent. The mortality data may also be selected for a cause of death related specifically to drug overdose, such as heroin, natural/semisynthetic opioid, other synthetic narcotics, cocaine, and psychostimulants with abuse potential. The second data set 108 may be selected for a specific geographic region or regions. The second data set 108 may be collected for a selected time interval. For example, a user may select mortality data for a specific county. For example a user may select mortality data for about five year period. For example, mortality data in Los Angeles County may be selected ranging from about 2016-2018. The second data source 110 may be the National Vital Statistics System or another appropriate data source providing drug overdose mortality data.

With reference now to FIG. 1C, of the illustrative drawings, there is shown a flowchart explaining an example method for performing the second step 156 of the process shown in FIG. 1A. The second step 156 involves obtaining user input selecting the collection interval 112 and the collection region 114 for the second data set 108, which comprises mortality data. The collection interval 112 may be at an annual, monthly, weekly, or daily level. The selected region(s) 114 may be at the country, state, county, city, or individual zip code level. In an example embodiment, a user may select mortality data 108 for all counties 114 within a state. The collection interval 112 for the mortality data may be at the annual level.

Referring back to FIG. 1A, in the example method shown, the processor then performs a third step 158 where it trains a health forecasting model 116 to describe a relationship 118 between the first 100 and second 108 data sets. The processor uses statistical methods, selected by a user, to empirically determine coefficients for inclusion in the health forecasting model 116. The relationship between the first data set 100 and second data set 108 may be a dependent relationship, such that the second data set 108 depends on the first data set 100. In some embodiments where the second data set 108 comprises drug overdose mortality data, the first data set 100 may predict the selected mortality rate. Given this relationship, a predictive model may be empirically obtained by training the health forecasting model 116 using the first data set and the second data set as training data sets and applying a statistical method, such as a Poisson regression method to the training data sets.

With reference now to FIG. 1D of the illustrative drawings, there is shown a flowchart explaining an example method for performing the third step 158 of the process shown in FIG. 1A. The third step 158 involves obtaining user input selecting features 120 for use in training the health forecasting mode 116. The third step 158 further involves performing user selected statistical training methods. User selected features 120 may include selected demographic characteristics and UDT rates having predictive value. The training methods may include a hyperparameter training 122, a regression model parameter training 124, and a nested cross validation 126, to confirm the model is accurate and has predictive value. The processor may perform some or all of these training methods to train the model parameters 116 to describe a relationship between the first data set 100 and the second data set 108. In some embodiments, model training may involve generating a model that predicts mortality based on selected predictor variable data, including UDT data and selected demographic data. In an alternative embodiment, model training may involve a reverse prediction, in which the model is trained to predict UDT data based on selected demographic features. This type of reverse prediction may be used to better understand drug use trends.

In an embodiment, a health forecasting model may be trained using a dual validation method. For example, two simple validations may be performed. A first validation may train a health forecasting model by applying a Poisson regression model to two training data sets including a predictor variable training data set and a dependent variable training data set at the county level for a period of about two years. A test data set from the year following the later year of the two year period may be selected. A first validation may involve assessing a prediction generated by the health forecasting model for the same year as the test data set against the test data set. Another validation may be performed for on offset time interval of about one year further in the future. Performance metrics such as Pearson correlation of measured and predicted mortality rates, Mean Square Error (MSE), Mean Absolute Deviation (MAD) and Absolute Percent Error (MAPE) may be used in the validations to train the health forecasting model.

### Example Embodiment for Generating Real-Time Drug Crisis Forecast

With reference now to FIG. 1E of the illustrative drawings, there is shown a flowchart explaining an example method for generating a real-time drug crisis forecast. A processor may first perform the method as shown in FIG. 1A to train the health forecasting model 116 to describe a relationship between the first data set 100 and the second data set 108. The processor then updates the first data set 100. The processor may obtain a user selection of a collection interval 130 to update the first data set 100. For example, a first data set 100 comprising UDT data for a selected county may be updated on a monthly interval 130. The processor then applies the model parameters 116 it developed to the updated first data set 100 to generate a predictive model based on the updated first data set 100 for a selected collection interval. The predictive model is a real-time drug crisis forecast 132. The real-time drug crisis forecast 132 may predict a drug crisis for a selected geographic region during a selected timeframe, based on user selections for the first data set 100, the second data set 108, and the updated first data set 100.

The processor may then perform a subprocess in which it performs a validation check 134 to test the accuracy and predictive value of the generated real-time drug crisis forecast 132. To perform the validation check, the processor may generate a real-time drug crisis forecast 132 which predicts mortality rates for a past period for a given region in which a second data set 108 comprising mortality data for that period and region is already available. Then, the processor may generate user selected performance metrics to measure the accuracy and predictive value of the model. For instance, these metrics may include a Pearson correlation of observed and predicted mortality rates, a Mean Square Error (MSE), a Mean Absolute Deviation (MAD), and/or a Mean Absolute Percent Error (MAPE). Statistical software, such as R statistical software version 4.0.2 (R Project for Statistical Computing) or another appropriate program or method may be used for data analysis. In some embodiments using R for data analysis, the glmr( ) function from the Ime4 v1.1-23 package may be used for estimating Poisson mixed models. In other embodiments, other appropriate estimate methods may be used.

In a graphical representation generation step 160, the processor may then generate one or more graphical representations 136 of the real-time drug crisis forecast 132. The graphical representations may take the form of a mapping, for instance showing regions of increasing drug use, a choropleth mapping, or a table showing comparative risk data. The processor may present the graphical representations to a user 136 in a dashboard. A user may select different parameters and filters to adjust the graphical representations 136 to view desired data and predictions specific to particular regional areas and time intervals.

With reference now to FIG. 1F of the illustrative drawings, there is shown a flowchart explaining an example method for generating graphical representations of a real-time drug crisis forecast based on obtained user input. A processor may perform the graphical representation generation step 160 of the process shown in FIG. 1E by first obtaining user input selecting one or more types of graphical representations. In some embodiments, one type of selected graphical representation may be a choropleth mapping. A choropleth mapping may present a shaded map to a user wherein areas of the map are shaded in proportion to a predicted variable. For example, geographic regions on a map may be shaded to indicate a predictive drug overdose mortality rate for a given time period. Regions shaded a darker color may indicate a higher rate of predicted mortality. Such a map may provide a visual for a user to quickly identify regions where a drug crisis is likely to cause significant stress and require significant resources.

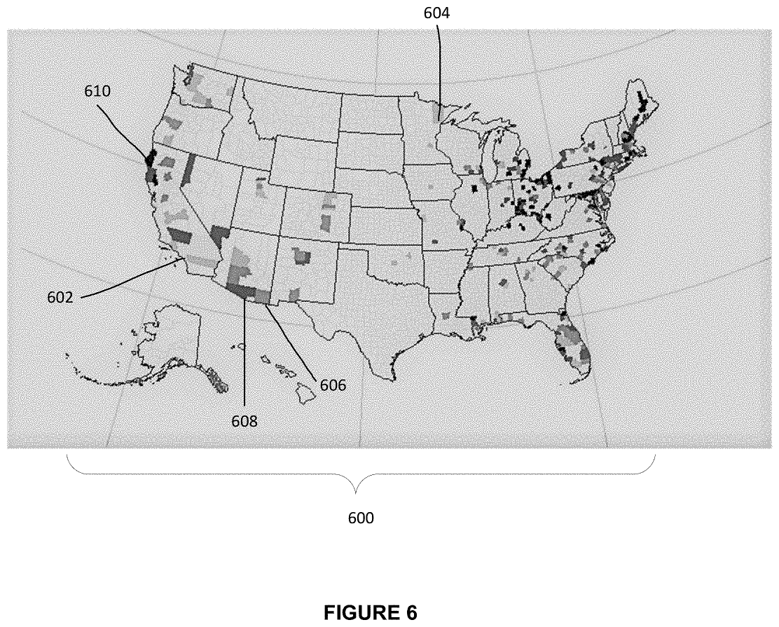

With reference now to FIG. 6, an example of a choropleth mapping for a real-time drug crisis forecast for the United States is shown. As shown in this embodiment, a selected region 600 comprises the entire United States. The choropleth mapping uses a light color to indicate the a lowest category 802 of predicted mortality within counties within states within the United States. A slightly darker color is used to indicate a low to medium predicted mortality rate category 604 for counties within states within the United States. A medium shade color is used to indicate a moderate predicted mortality rate category 606 for counties within states within the United States. A darker shade is used to indicate a moderate to severe predicted mortality rate category 608 for counties within states within the United States. The darkest shade is used to indicate a severe predicted mortality rate category 610 for counties within states within the United States. In this example embodiment, the choropleth mapping is determined at a county level and includes selected counties within the United States.

Referring back to FIG. 1F, in another embodiment, one type of selected graphical representation may be a table. A table may be used to present data associated with several user selected regions for several user selected timeframes, such that a user can analyze the comparative risk between regions for a given time period. Alternatively, a user may configure such a table to determine the increase in risk of a drug crisis over time within a selected region.

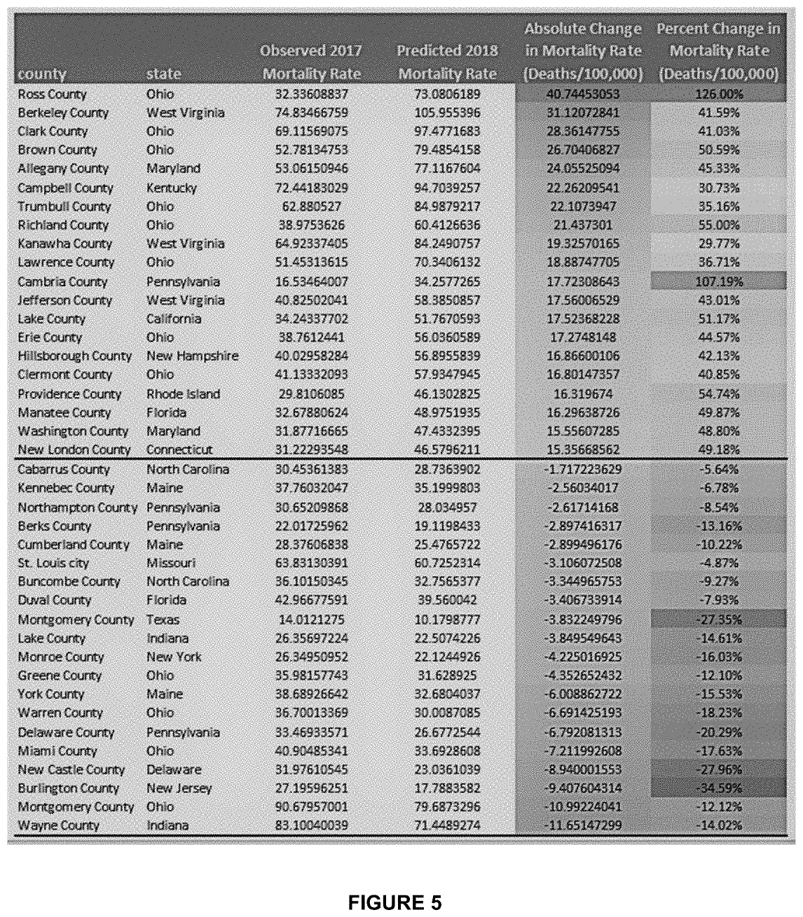

Referring now to FIG. 5, an example of a real-time drug crisis forecast table is shown. In this example embodiment, data is shown for selected counties within the United States. The counties are listed in the left most column. Observed mortality data comprising a second data set 108 is shown in the third column. This mortality data is provided at the county level for an annual period, the year 2017. Then, in the next column, predicted mortality data, based on a generated real-time drug crisis forecast as described in FIG. 1E is presented at the county level for the year 2018. This allows the user to compare past indications of a drug crisis with current indications of a drug crisis. Additionally, comparative parameters follow in the next two columns. In this example, the second to last column presents an absolute change in mortality rate per 100,000 persons between the observed 2017 rates and the predicted 2018 rates, on a county level. The last column presents a percent change in mortality rate per 100,000 persons on a county level.

Referring back to FIG. 1F, in other embodiments, graphical representations 136 may include charts, heat maps, plots, and other user selected graphical tools. After the processor obtains user input selecting the type or types of graphical representations, the process may then obtain user input selecting the geographic regions and time intervals for inclusion in the representations. The geographical regions may be identified on the country, state, county, city, and zip code level. A user may select one or more geographical regions or subregions for inclusion in a graphical representation. For example, In some embodiments as shown in FIG. 5, the processor obtains user input selecting several counties for inclusion in the graphical representation of the real-time drug crisis forecast. In another embodiment, as shown in FIG. 6, the processor obtains user input selecting counties for inclusion in the graphical representation but also showing state and country boundaries. The user selected time intervals may be at the annual, quarterly, monthly, weekly, or daily level. For example, in an embodiment, a user may select a time interval for a period of about three months. For example, a user may select a time interval of from about June of 2019 to August of 2019. A user may select one or more time intervals for inclusion in the graphical representation. For example, as shown in FIG. 5, two annual time intervals for two different years are shown. In another embodiment, as shown in FIG. 6, one time interval at the annual level is shown.

### Example Embodiment for Generating Resource Deployment Plan

With reference now to FIG. 1G of the illustrative drawings, there is shown a flowchart explaining an example method for generating a resource deployment plan 142 based on a generated real-time drug crisis forecast 132. A processor may perform the health forecasting model 116 training method as shown in FIG. 1A. Then, in the next step 162, a processor may obtain a third data set 138 from a third data source 140. The third data set 138 may comprise drug crisis response resource data. The third data source 140 may include government agencies at the state, local, and national levels, including law enforcement agencies such as police departments and emergency services agencies, such as fire departments. The third data sources 140 may also include legislative bodies, scientific research bodies, community organizations, the medical community, and other relevant sources.

With reference now to FIG. 1H of the illustrative drawings, there is shown a flowchart explaining an example method for obtaining user input in selecting a third data set 138 from a third data source 140. The processor may first obtain user input regarding the type or types of drug crisis response resource data. For example, In some embodiments, a user may select a data set showing the number of patients rehabilitation centers may accommodate. In another example embodiment, a user may select a data set showing the availability, cost, and efficacy of overdose-reversing drugs. In another embodiment, a user may select a data set showing the availability of emergency response resources, such as the number of EMTs in a given region. A user may select one or more types of drug crisis response resource data for inclusion in the third data set 138.

Next the processor may obtain user indications of collection regions and collection intervals for the third data set 138. Collection regions may be at the national, state, county, city or zip code level. Collection intervals may be at the annual, quarterly, monthly, weekly, or even daily levels. For instance, in an example embodiment, a user may select a third data set 138 comprising the availability of fire department resources in Los Angeles County on a monthly basis. For example, a user may select an interval of a range of about one month. For example, a user may select data for Los Angeles County for the month of October in the year 2020.

Referring back to FIG. 1G, the processor may then perform a real-time drug crisis forecast generation method as shown in FIG. 1E. The process may then generate a resource deployment plan 142 in a resource deployment plan generation step 164. A resource deployment plan may include an efficient allocation of existing drug crisis response resource. A resource deployment plan may also including identification of missing or needed resource. A resource deployment plan may include funding allocations for the development of needed resources. A resource allocation plan may also include an efficient resource sharing between geographical regions based on respective need.

With reference now to FIG. 1I of the illustrative drawings, there is shown a flowchart explaining an example method for generating a resource deployment plan 142 as in step 164 of the resource deployment method of FIG. 1G. The processor may first obtain user input selection a region or regions 144 and a subregion or subregions 146 of interest for resource deployment purposes. In an example embodiment, a user may select a state as a region of interest for resource deployment purposes. A user may select several counties within the state as subregions of interest for resource deployment purposes. The processor may then obtain a third data set showing available resources at the county level within the state of interest.

The processor may then perform a comparative risk assessment 148 for the user selected subregions. For example, In some embodiments, a user may be a state government agency. The agency may be interested in a comparative risk assessment for counties within the state. The agency may select several counties as subregions of interest. The processor may then obtain resource availability data at the county level. The processor may then perform a comparative risk assessment by considering both the predicted mortality rate for the selected subregions during a selected time interval as well as the available response resources. The processor may return a drug crisis forecast that indicates predicted mortality for a selected county during a selected time period is high. The processor may return an indication, based on the resource availability data, that the high risk county also lacks needed resource to respond to a crisis. The processor may then perform a comparative risk assessment by generating a real-time drug crisis forecast for another county within the state. The processor may return a forecast indicating the second county presents a comparatively low risk of predicted mortality within the selected time frame. The process may then return an indication, based on the resource availability data, that the second county has a surplus of available response resources.

The processor may then perform an efficient resource allocation 150 based on the comparative risk assessment 148. For example, in the embodiment described above, the processor may return to the state agency an efficient resource allocation plan based on the comparative risk for the two selected subregion counties. The processor may return a resource allocation that proposes a sharing of resources between the selected counties because the first region lacks resources and anticipated a high predicted mortality rate while the second region has an abundance of resources but does not anticipate a high mortality rate.

Referring back to FIG. 1G, the processor may then communicate a resource deployment plan 142 to relevant organizations. Organizations may include law enforcement agencies, governing bodies, legislative bodies, medical communities, community organizations, scientific research and development organizations, and other relevant organizations that may be in a position to study, assess, or respond to a predicted drug crisis.

### Example Embodiments for Real Time Drug Crisis Prediction System

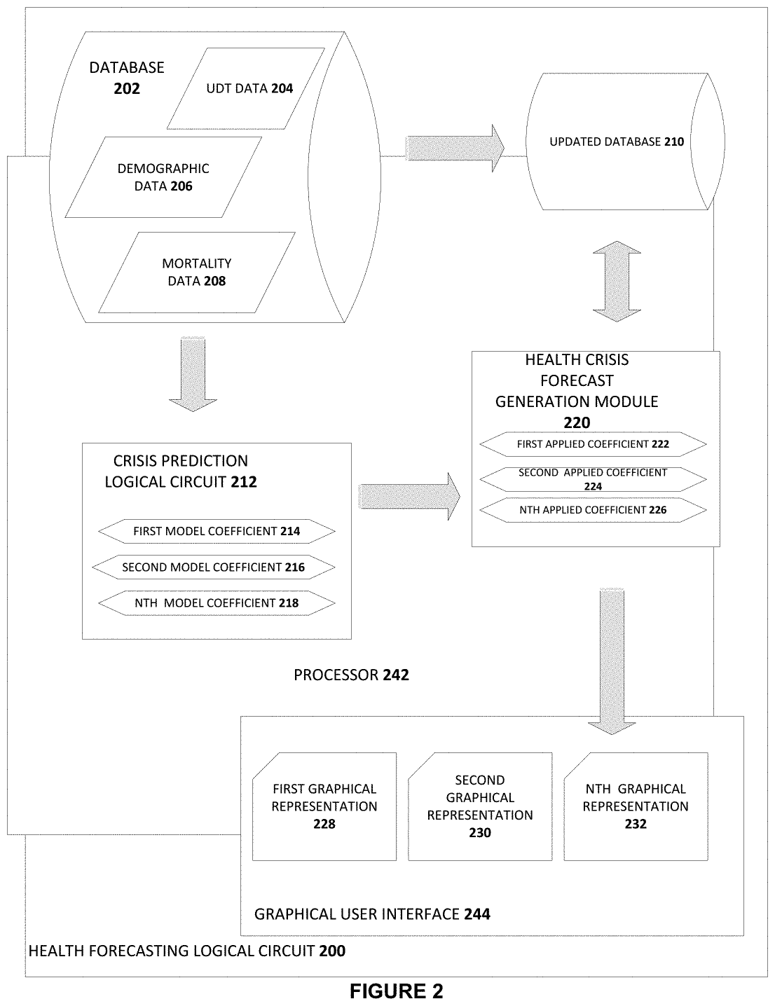

With reference now to FIG. 2 of the illustrative drawings, there is shown an example health crisis prediction system. A health crisis prediction system may be in include a health forecasting logical circuit 200 comprising a processor 242 and a graphical user interface 244. The processor 242 in a health crisis prediction system may further comprise a database 202, an updated database 210, a crisis prediction logical circuit 212, and health crisis forecast generation module 220. The graphical user interface 244 in a health crisis prediction system may comprise several graphical representations 228, 230, and 232 of a real-time health crisis forecast.

A database 202 may include several data sets. In an example embodiment, as shown in FIG. 2, the data sets may include UDT data 204, demographic data 206, and mortality data 208. An updated data base 210 may also include similar types of data and may be updated on a selected time interval. The database 202 may also include data sets not shown in FIG. 2. For example, crime lab seizure data, emergency room visitation data, suboxone/naloxone prescription rates, methadone prescription rates, buprenorphine prescription rates, monthly unemployment rates may be included in the database in embodiments of the present disclosure. All data included in the database 202 may have spatial and temporal dimensions. For instance data may be collected at the country, state, county, city, and zip code level. Data may be collected and updated at annual, quarterly, monthly, weekly or daily intervals. The data time interval may be a fixed or random factor. Consideration of the time interval as a fixed or random factor may contribute to improved prediction of drug crises.

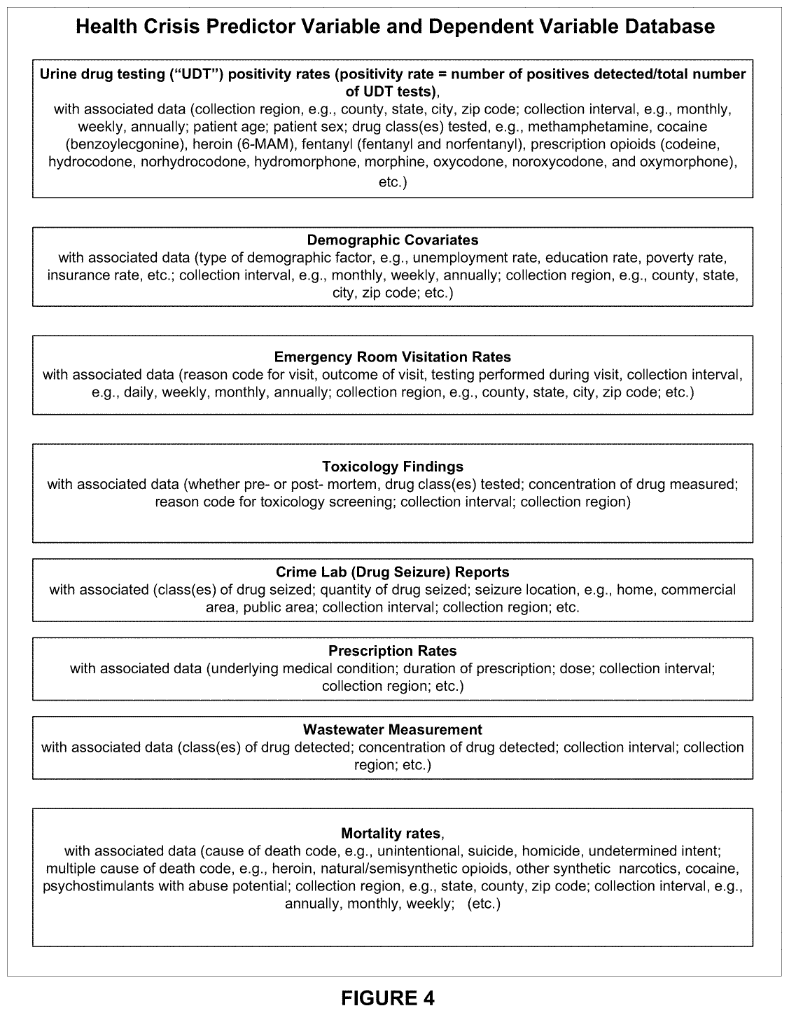

With reference now to FIG. 4 of the illustrative drawings, there is shown an example of a health crisis predictor and dependent variable database for use in health crisis prediction systems and methods. The database as shown in FIG. 4 may be used as a database 202 or updated database 210 in the health crisis prediction system of FIG. 2.

Referring back to FIG. 2, the crisis prediction logical circuit 212 of the health crisis prediction system may include several model parameters 214, 216, and 218. Several statistical methods may be used to generate the model parameters. The crisis prediction logical circuit 212 may comprise a logistic regression model. Several statistical methods may be used to product the logistic regression model. For example, Poisson regression, negative binomial regression, logistic regression (dichotomized mortality, high/low, etc.), regression trees, random forest, regularized regression (e.g., lasso, enet), and non-linear prediction (e.g., generalized additive models, etc.), may, among other methods, be used to produce the logistic regression model used to train the model parameters.

The health crisis prediction system may further comprise a health crisis forecast generation module 212 which may include several applied coefficients 222, 224, 226 which are included in a health forecasting model 116. The applied coefficients may be included in the health forecasting model applied to the updated data sets from the updated database 210 to form a health crisis forecast as described in the method of FIG. 1E.

The health crisis prediction system may further comprise a graphical user interface 244, which may include one or more graphical representations 228, 230, and 232 of the health crisis forecast. The graphical representations may be generated according to the method of FIG. 1F. The graphical representations may comprise tables, maps, charts, and other graphics with user selectable filters and parameters. Examples of graphical representations are shown in FIGS. 5 and 6 and described herein.

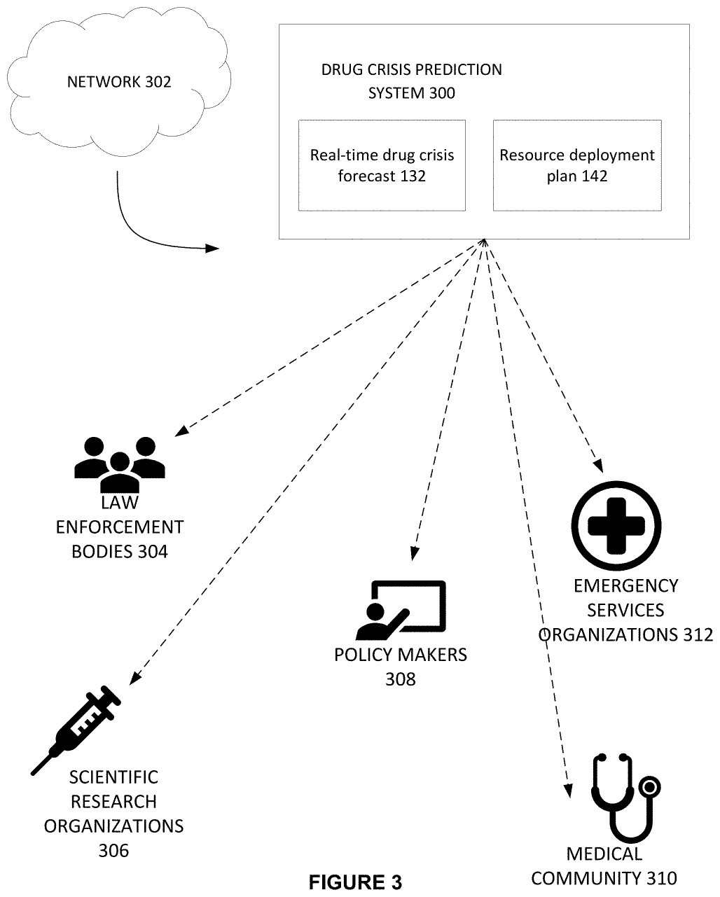

With reference now to FIG. 3 of the illustrative drawings, there is shown an example resource deployment communication system. A resource deployment communication system may comprise a network 302, a drug crisis prediction system 300 and several network participants including law enforcement bodies 304, scientific research organizations 306, policy makers 308, medical communities 310, and emergency services organizations 312. The drug crisis prediction system 300 may product a real-time drug crisis forecast 132 according to the method of FIG. 1E. The drug crisis prediction system may product a resource deployment plan 142 according to the method of FIG. 1G. The network participants 304, 306, 308, 310, and 312 may receive the real-time drug crisis forecast 132 and the resource deployment plan 142 over a network. The network participants 304, 306, 308, 310, and 312 may also receive any updates to the real-time drug crisis forecast 132 and the resource deployment plan 142 over the network.

### Alternative Embodiments

In addition to the above embodiments, alternative embodiments may also be beneficial under certain circumstances.

For example, in an alternative embodiment, machine learning methods may be used to train model parameters and develop modes. Machine learning methods may include Support Vector Machines and Neural networks.

In another embodiment, it may be desirable to study trends and generate a prediction for a specific geographic region having specific needs. Additional predictor variable data, including demographic data, may be obtained for a specific region to generate a customized model.

In some embodiments, time series modeling techniques may be desirable. Time series modeling techniques may include ARIMA models, spatio-temporal modeling, GEE methods, and other time series modeling techniques.

In some embodiments, additional UDT analytes may be desirable. Additional UDT analytes may include fentanyl analogs and benzodiazepines.

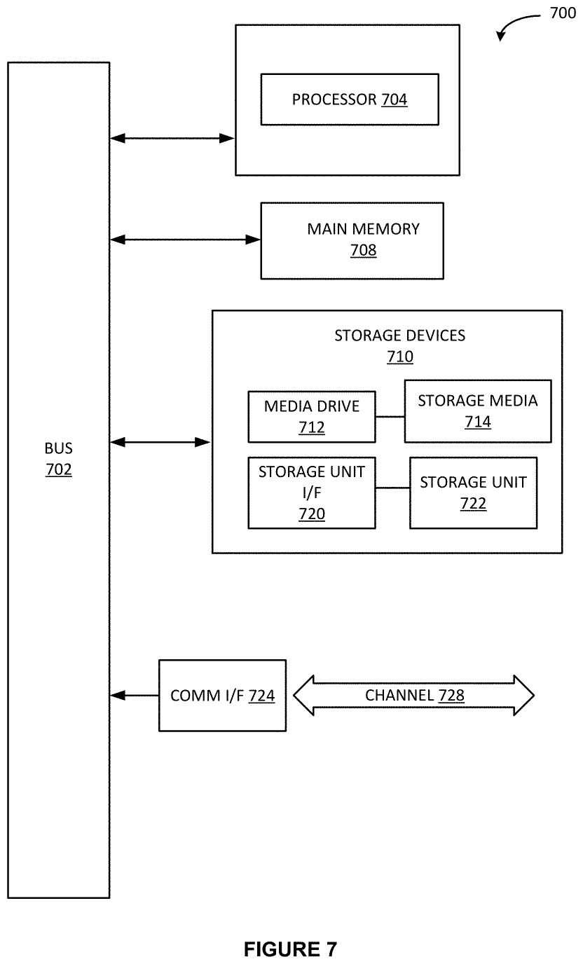

### Software Elements for Use in Health Forecasting Logical Circuit

Where components or components of the application are implemented in whole or in part using software, in one embodiment, these software elements can be implemented to operate with a computing or processing component capable of carrying out the functionality described with respect thereto. One such example computing component is shown in FIG. 7 which may be used to implement various features of the system and methods disclosed herein. Various embodiments are described in terms of this example-computing component 700. After reading this description, it will become apparent to a person skilled in the relevant art how to implement the application using other computing components or architectures.

Referring now to FIG. 7, computing component 700 may represent, for example, computing or processing capabilities found within a self-adjusting display, desktop, laptop, notebook, and tablet computers; hand-held computing devices (tablets, PDA's, smart phones, cell phones, palmtops, etc.); workstations or other devices with displays; servers; or any other type of special-purpose or general-purpose computing devices as may be desirable or appropriate for a given application or environment. For example, computing component 700 may be one embodiment of the data acquisition and control component of FIG. 7, a GED, and/or one or more functional elements thereof. Computing component 700 might also represent computing capabilities embedded within or otherwise available to a given device. For example, a computing component might be found in other electronic devices such as, for example navigation systems, portable computing devices, and other electronic devices that might include some form of processing capability.

Computing component 700 might include, for example, one or more processors, controllers, control components, or other processing devices, such as a processor 704. Processor 704 might be implemented using a general-purpose or special-purpose processing engine such as, for example, a microprocessor, controller, or other control logic. In the illustrated example, processor 704 is connected to a bus 702, although any communication medium can be used to facilitate interaction with other components of computing component 700 or to communicate externally.

Computing component 700 might also include one or more memory components, simply referred to herein as main memory 708. For example, preferably random access memory (RAM) or other dynamic memory might be used for storing information and instructions to be executed by processor 704. Main memory 708 might also be used for storing temporary variables or other intermediate information during execution of instructions to be executed by processor 704. Computing component 700 might likewise include a read only memory (“ROM”) or other static storage device coupled to bus 702 for storing static information and instructions for processor 704.

The computing component 700 might also include one or more various forms of storage device 710, which might include, for example, a media drive 712 and a storage unit interface 720. The media drive 712 might include a drive or other mechanism to support fixed or removable storage media 714. For example, a hard disk drive, a solid state drive, a magnetic tape drive, an optical disk drive, a compact disc (CD) or digital video disc (DVD) drive (R or RW), or other removable or fixed media drive might be provided. Accordingly, storage media 714 might include, for example, a hard disk, an integrated circuit assembly, magnetic tape, cartridge, optical disk, a CD or DVD, or other fixed or removable medium that is read by, written to or accessed by media drive 712. As these examples illustrate, the storage media 714 can include a computer usable storage medium having stored therein computer software or data.

In alternative embodiments, storage device 710 might include other similar instrumentalities for allowing computer programs or other instructions or data to be loaded into computing component 700. Such instrumentalities might include, for example, a fixed or removable storage unit 722 and an interface 720. Examples of such storage units 722 and interfaces 720 can include a program cartridge and cartridge interface, a removable memory (for example, a flash memory or other removable memory component) and memory slot, a PCMCIA slot and card, and other fixed or removable storage units 722 and interfaces 720 that allow software and data to be transferred from the storage unit 722 to computing component 700.

Computing component 700 might also include a communications interface 724. Communications interface 724 might be used to allow software and data to be transferred between computing component 700 and external devices. Examples of communications interface 724 might include a modem or softmodem, a network interface (such as an Ethernet, network interface card, WiMedia, IEEE 802.XX or other interface), a communications port (such as for example, a USB port, IR port, RS342 port Bluetooth® interface, or other port), or other communications interface. Software and data transferred via communications interface 724 might typically be carried on signals, which can be electronic, electromagnetic (which includes optical) or other signals capable of being exchanged by a given communications interface 724. These signals might be provided to communications interface 724 via a channel 728. This channel 728 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 708, storage unit 720, media 714, and channel 728. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 700 to perform features or functions of the present application as discussed herein.

It should be understood that the various features, aspects and functionality described in one or more of the individual embodiments are not limited in their applicability to the particular embodiment with which they are described. Instead, they can be applied, alone or in various combinations, to one or more other embodiments, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A health forecasting system comprising:
a health forecasting logical circuit and a graphical user interface, the health forecasting logical circuit comprising a processor; and
a non-transient memory with computer executable instructions embedded thereon, the computer executable instructions configured to cause the processor to:
obtain a first data set from a first data source, wherein the first data set is selected from a group consisting of: positive drug test rate for one or more controlled substance, crime lab seizure data, emergency room visitation data, prescription rates, and demographic data for a regional population;
obtain a second data set from a second data source, wherein the second data set comprises mortality data for a regional population; and
train, with a crisis prediction logical circuit, a health forecasting model, wherein the health forecasting model describes a relationship between the second data set and the first data set by a dual validation approach including temporally offset data sets.

2. The system of claim 1,
wherein the health forecasting model comprises a logistic regression, a gradient-boosted decision tree, or a cognitive neural network.

3. The system of claim 1, wherein the computer executable instructions further cause the processor to:
update the first data set from the first data source on a selected time interval;
apply the health forecasting model to the updated first data set;
generate a real-time drug crisis forecast based on the application of the health forecasting model to the first data set for the selected time interval; and
generate one or more graphical data representations based on the generated drug crisis forecast, wherein the graphical data representations are based on user-selected model data structures.

4. The system of claim 3, wherein the computer executable instructions further cause the processor to:
obtain a third data set from a third data source comprising available drug crisis response resources for a regional population; and
generate a resource deployment plan based on the drug crisis forecast and the available drug crisis response resources in the selected geographical region.

5. The system of claim 4, wherein the system generates comparative drug crisis forecasts for multiple selected geographic regions such that a comparative risk assessment may be performed and a resource allocation plan for the selected geographic regions may be generated based on the comparative risk assessment.

6. The system of claim 1, wherein the first data set comprises urine drug testing (“UDT”) data.

7. The system of claim 1, wherein the first data set comprises demographic data selected from a group consisting of: unemployment rates, education rates, poverty rates, and insurance rates.

8. The system of claim 6, wherein the UDT data is collected at the county level for a regional population and is updated on a monthly timeframe.

9. The system of claim 7 wherein the demographic data is collected at the county level for a regional population and is updated on a monthly timeframe.

10. The system of claim 1, wherein the processor trains the health forecasting model to describe the relationship between the first and second data sets using at least one of the following regression methods: Poisson regression, negative binomial regression, logistic regression, regression trees, random forest, regularized regression, and non-linear prediction.

11. The system of claim 3, wherein the user-selected model data structures provide a comparative risk assessment and include at least one of the following: a choropleth map and a table ranking counties by determined risk level.

12. A method for mitigating the localized impact of a health crisis, the method comprising:
obtaining, with a graphical user interface, a first data set from a first data source, wherein the first data set is selected from a group consisting of: positive drug test rate for one or more controlled substance, crime lab seizure data, emergency room visitation data, prescription rates, and demographic data for a regional population;
obtaining with a graphical user interface a second data set from a second data source, wherein the second data set comprises mortality data for a regional population;
training, with a crisis prediction logical circuit, a health forecasting model, wherein the health forecasting model describes a relationship between the second data set and the first data set, by a dual validation approach including temporally offset data sets; updating the first data set from the first data source on a selected time interval;
applying the health forecast model to the updated first data set;
generating a real-time health crisis forecast based on the application of the health crisis model to the updated first data set for the selected time interval;
generating one or more graphical data representations based on the generated health crisis forecast, wherein the graphical data representations are based on user-selected model data structures.

13. The method of claim 12, wherein the health forecast model comprises a logistic regression model, a gradient-boosted decision tree, or a cognitive neural network.

14. The method of claim 12, wherein the first data set comprises UDT data.

15. The method of claim 12, wherein the first data set comprises demographic data selected from a group consisting of: unemployment rates, education rates, poverty rates, and insurance rates.

16. The method of claim 14, wherein the UDT data is collected at the county level for a regional population and is updated on a monthly timeframe.

17. The method of claim 15 wherein the demographic data is collected at the county level for a regional population and is updated on a monthly timeframe.

18. The method of claim 12, wherein the processor trains the health forecasting model to describe the relationship between the first and second data sets using at least one of the following regression methods: Poisson regression, negative binomial regression, logistic regression, regression trees, random forest, regularized regression, and non-linear prediction.

19. The method of claim 12, wherein the user-selected model data structures provide a comparative risk assessment and include at least one of the following: a choropleth map and a table ranking counties by determined risk level.

20. A health forecasting system comprising:
a health forecasting logical circuit and a graphical user interface, the health forecasting logical circuit comprising a processor; and
a non-transient memory with computer executable instructions embedded thereon, the computer executable instructions configured to cause the processor to:
obtain a first data set from a first data source, wherein the first data set is selected from a group consisting of: positive drug test rate for one or more controlled substance, crime lab seizure data, emergency room visitation data, prescription rates, and demographic data for a regional population;
obtain a second data set from a second data source, wherein the second data set comprises mortality data for a regional population; and
train, with a crisis prediction logical circuit, a health forecasting model, wherein the health forecasting model describes a relationship between the second data set and the first data set;
wherein the health forecasting model comprises a logistic regression model, a gradient-boosted decision tree, or a cognitive neural network;
update the first data set from the first data source on a selected time interval;
apply the health forecast model to the updated first data set;
generate a real-time health crisis forecast based on the application of the health forecasting model to the first data set for the selected time interval;
generate one or more graphical data representations based on the generated health crisis forecast, wherein the graphical data representations are based on user-selected model data structures.
